(19) United States
(12) Patent Application Publication
(10) Pub. No.: US 2005/0019395 A1
(43) Pub. Date: Jan. 27, 2005

(54) Formulations of amlodipine maleate

(76) Inventors: Gabor Pragai, Debrecen (HU); Eva Orosz, Debecen (HU); Judit Szilagyi, Debrecen (HU); Edit Nagy, Debrecen (HU); Lidia Ban, Debrecen (HU)

Correspondence Address: KENYON & KENYON, ONE BROADWAY, NEW YORK, NY, 10004, US

(21) Appl. No.: 10/823,802

(22) Filed: Apr. 12, 2004

Related U.S. Application Data
(60) Provisional application No. 60/462,813, filed on Apr. 14, 2003.

(51) Int. Cl. A61K 31/455; A61K 9/20

(52) U.S. Cl. 424/464; 514/355

(57) ABSTRACT

The present invention provides improved, more stable formulations of amlodipine maleate where the formulations comprise from none to a minimal amount of magnesium. Such stable formulations show decreased production of the impurity amlodipine aspartate. Accordingly, the present invention provides formulations of amlodipine maleate comprising lubricants such as sodium stearyl fumarate, dimeticone, macrogol 6000, hydrogenated castor oil, and stearic acid. Methods of making and using the improved formulations are also provided.

This application claims the benefit of U.S. Provisional Patent Application No. 60/462,813, filed Apr. 14, 2003, which is incorporated herein by reference in its entirety.

### FIELD OF THE INVENTION

The present invention relates to a process for preparing improved formulations of amlodipine maleate as well as pharmaceutical compositions comprising the improved formulations of amlodipine maleate where the improved formulations of amlodipine maleate comprise from none to a minimal amount of magnesium.

### BACKGROUND OF THE INVENTION

Amlodipine is a calcium channel blocker approved in the United States for the treatment of certain types of hypertension and sold under the tradename NORVASC®. NORVASC®contains the besylate salt of amlodipine. When developing NORVASC®, a switch was made by the manufacturer from the original maleate salt of amlodipine to the besylate salt. The switch to the besylate salt was made after the manufacturer encountered stability and tableting problems with the maleate salt. These problems were subsequently determined to be attributable to a biologically-active degradation product, then referred to as UK-57,269, that arises during synthesis and production of the maleate salt. UK-57,269 is now known to be amlodipine aspartate.

It would be desirable to have other formulations of amlodipine available besides the besylate salt. In particular, it would be desirable to have a formulation of the maleate salt of amlodipine that does not contain significant amounts of amlodipine aspartate and that does not degrade during long term storage to produce significant amounts of amlodipine aspartate. There is a need for stable formulations of amlodipine maleate that do not degrade into amlodipine aspartate.

International Patent Publication WO 02/053134 states that a more stable amlodipine maleate pharmaceutical composition is provided when formulated with a pH within the range of 5.5 to 7, when measured as a 20% aqueous slurry. The stability can also be aided by making the pharmaceutical composition from amlodipine maleate particles having an average particle size of greater than 20 μm, preferably greater than 100 μm. U.S. Patent Application No. 2003/0027848 states that amlodipine maleate can be stabilized by adding to the composition an acid having a pK₁ of greater than 0.5 which is present in an amount sufficient to prevent the formation of the aspartate impurity.

U.S. Pat. No. 5,006,344 discloses stable tablets of fosinopril employing either sodium stearyl fumarate or hydrogenated vegetable oil as lubricants.

### SUMMARY OF THE INVENTION

The present invention provides improved stable formulations of amlodipine maleate where the formulations comprise from none to a minimal amount of magnesium, particularly from none to a minimal amount of magnesium stearate. The present inventors have determined that the stability of certain formulations of amlodipine maleate is markedly improved when the amount of magnesium in such formulations is reduced or, preferably, eliminated. Such stable formulations show decreased production of the impurity amlodipine aspartate.

In particular, the present inventors have determined that the addition of lubricants containing magnesium to amlodipine maleate formulations is to be avoided. Accordingly, in certain aspects, the present invention is directed to formulations of amlodipine maleate comprising lubricants where the lubricant does not contain magnesium. In other aspects, the formulations comprise a minimal amount by weight of a magnesium-containing lubricant, e.g., less than 1% magnesium stearate, preferably less than 0.5% magnesium stearate, even more preferably less than 0.1% magnesium stearate.

Accordingly, the present invention includes formulations of amlodipine maleate comprising:
a therapeutically effective amount of amlodipine maleate,
a binder,
a diluent,
a disintegrant, and
a lubricant that does not contain magnesium.

Pharmaceutical compositions containing the formulations of the invention are also provided. Such compositions are preferably in the form of tablets.

Suitable binders include microcrystalline cellulose, modified celluloses, and povidone.

Suitable diluents include calcium hydrogen phosphate (CaHPO₄), anhydrous; lactose; and mannitol.

Suitable disintegrants include sodium starch glycollate (type A), sodium starch glycollate (type B), and crospovidone.

Suitable lubricants that do not contain magnesium include sodium stearyl fumarate, dimeticone, macrogol 6000, hydrogenated castor oil, and stearic acid.

Optionally, the formulations may include other excipients in addition to binders, diluents, disintegrants, and lubricants. For example, the formulations may include colorants or taste masking agents.

In preferred embodiments, the present invention provides formulations of amlodipine maleate comprising:
a therapeutically effective amount of amlodipine maleate,
microcrystalline cellulose,
calcium hydrogen phosphate (CaHPO₄), anhydrous,
sodium starch glycollate (type B), and
a lubricant that does not contain magnesium.

The present invention also provides methods of making formulations of amlodipine maleate where the methods comprise combining:
a therapeutically effective amount of amlodipine maleate,
a diluent,
a binder,
a disintegrant, and
a lubricant that does not contain magnesium,
where the resulting formulation of amlodipine maleate formed by so combining contains less than 0.5% amlodipine aspartate.

The present invention also includes a method of treating and/or preventing hypertension, angina, or heart failure comprising administering to a patient in need thereof a therapeutically effective amount of a pharmaceutical composition comprising:
amlodipine maleate,
a diluent,
a binder,
a disintegrant, and
a lubricant that does not contain magnesium,
where the pharmaceutical composition comprises less than 0.5% amlodipine aspartate.

### DETAILED DESCRIPTION OF THE INVENTION

When formulations of amlodipine maleate were produced with lubricants containing magnesium, e.g., magnesium stearate, certain impurities were observed during stability testing at 40° C./75% relative humidity. Two main degradation products were observed during the stability study:
(1) Aromatic impurity: 3-ethyl-5-methyl 2-[(2-aminoethoxy)methyl]-4-(2-chlorophenyl)-6 methyl-pyridine-3,5-dicarboxylate. This impurity is called Impurity D in the European Pharmacopoeia. 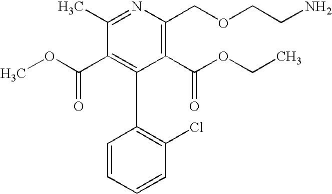
(2) Amlodipine aspartate: 3-ethyl-5-methyl 2-[{2-(N-succinyl)-aminoethoxy}methyl]-4-(2-chlorophenyl)-6 methyl-1,4-dihydropyridine-3,5-dicarboxylate

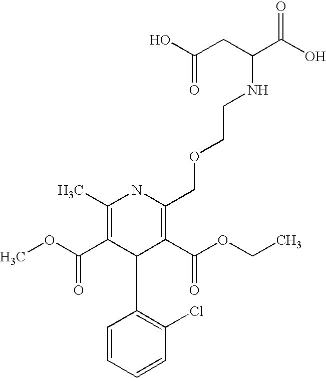

The presence of Impurity D is not a critical issue as according to the literature (e.g. Beresford et al, Pfizer Central Research, Xenobiotica, 1988, Vol. 18, No.2 245-254) the initial metabolic transformation common to 1,4-dihydropyridine based calcium channel blockers such as amlodipine involves oxidation of the dihydropyridine moiety to the aromatic pyridine analogue, i.e., Impurity D in the case of amlodipine. Amlodipine aspartate is produced in the reaction of amlodipine and maleic acid during a Michael addition. Amlodipine aspartate is not a qualified impurity of amlodipine so its amount should not exceed the 0.5% qualification threshold of the relevant ICH guideline (ICH Topic Q3B (R) Impurities in New Drug Products). During stability testing, formulations similar to the preferred embodiments described herein, but with lubricants containing magnesium rather than the non-magnesium-containing lubricants of the preferred embodiments, the amount of amlodipine aspartate exceeded the 0.5% level in 2 months at 40° C./75% relative humidity.

Preferred formulations of the present invention comprise, by weight:

| | |
|---|---|
| amlodipine maleate | about 2%-4%% |
| microcrystalline cellulose | about 50%-70% |
| calcium hydrogen phosphate (CaHPO₄), anhydrous | about 25%-35% |
| sodium starch glycollate (type B) | about 1%-4% |
| a lubricant that does not contain magnesium. | about 1%-3% |

Other formulations may contain slightly less microcrystalline cellulose and may comprise:

| | |
|---|---|
| amlodipine maleate | about 2%-4%% |
| microcrystalline cellulose | about 40%-70% |
| calcium hydrogen phosphate (CaHPO₄), anhydrous | about 25%-50% |
| sodium starch glycollate (type B) | about 1%-4% |
| a lubricant that does not contain magnesium. | about 1%-3% |

Other formulations may contain somewhat more lubricant and may comprise:

| | |
|---|---|
| amlodipine maleate | about 2%-4%% |
| microcrystalline cellulose | about 40%-70% |
| calcium hydrogen phosphate (CaHPO₄), anhydrous | about 25%-50% |
| sodium starch glycollate (type B) | about 1%-4% |
| a lubricant that does not contain magnesium. | about 1%-7% |

A particularly preferred formulation of the present invention comprises, by weight:

| | |
|---|---|
| amlodipine maleate | 3.21% |
| microcrystalline cellulose | 59.79-63.79% |
| calcium hydrogen phosphate (CaHPO₄), anhydrous | 30.00% |
| sodium starch glycollate (type B) | 2-4% |
| a lubricant that does not contain magnesium. | 1-3% |

Especially preferred formulations of the present invention comprise not more than 0.5% amlodipine aspartate after storage for two months at 40° C./75% relative humidity. In other embodiments, the formulations of the present invention comprise less than 5%, preferably less than 3%, and even more preferably less than 2% amlodipine aspartate after storage at 100° C. for 24 hours.

In addition to the active ingredient amlodipine maleate, various excipients may be used in the formulations of the present invention. Binders, i.e., excipients whose functions include helping to bind the active ingredient and other excipients together after compression of the formulations into tablets, may be included in the formulations. Binders that may be used in the present invention include, for example, acacia, alginic acid, carbomer (e.g., carbopol), carboxymethylcellulose sodium, dextrin, ethyl cellulose, gelatin, guar gum, hydrogenated vegetable oil, hydroxyethyl cellulose, hydroxypropyl cellulose (e.g., KLUCEL®), hydroxypropyl methyl cellulose (e.g., METHOCEL®), liquid glucose, maltodextrin, methylcellulose, polymethacrylates, povidone (e.g., KOLLIDON®, PLASDONE®), pregelatinized starch, sodium alginate, microcrystalline cellulose, modified cellulose, and starch.

The dissolution rate of a compacted solid pharmaceutical composition in the patient's stomach can be increased by the addition of a disintegrant to the composition. Disintegrants that may be used in the present invention include, for example, alginic acid, carboxymethylcellulose calcium, carboxymethylcellulose sodium (e.g., AC-DI-SOL®, PRIMELLOSE®), croscarmellose sodium, crospovidone (e.g., KOLLIDON®, POLYPLASDONE®), guar gum, methyl cellulose, microcrystalline cellulose, polacrilin potassium, powdered cellulose, pregelatinized starch, sodium alginate, sodium starch glycolate (type A or B), and starch.

When a dosage form such as a tablet is made by compaction of a powdered composition, the composition is subjected to pressure from punches and a die. Some excipients and active ingredients have a tendency to adhere to the surfaces of the punches and die, which can cause the product to have pitting and other surface irregularities. A lubricant can be added to the formulations of the present invention to reduce adhesion and ease release of the product from the punches and die. Lubricants that may be used in the present invention contain little or no magnesium and may include, for example, colloidal silicon dioxide, powdered cellulose, starch, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, macrogol 6000, dimeticone, stearic acid, and talcum.

Flavoring agents and flavor enhancers make the dosage form more palatable to the patient. Common flavoring agents and flavor enhancers for pharmaceutical products that can be included in the formulations of the present invention include for example maltol, vanillin, ethyl vanillin, menthol, citric acid, fumaric acid, ethyl maltol, and tartaric acid.

Formulations of the present invention can also be dyed using any pharmaceutically acceptable colorant to improve their appearance and/or facilitate patient identification of the product and unit dosage level.

In certain embodiments of the preferred formulation, the lubricant that does not contain magnesium is selected from the group consisting of: sodium stearyl fumarate, dimeticone, macrogol 6000, hydrogenated castor oil, colloidal silicon dioxide, talcum, and stearic acid. In certain embodiments, the lubricant is sodium stearyl fumarate at 0.5%-3% by weight; preferably 1%-2% by weight. In certain embodiments, the lubricant is hydrogenated castor oil at 1%-3% by weight; preferably 2%. In certain embodiments, the lubricant is colloidal silicon dioxide at about 3% by weight. In certain embodiments, the lubricant is talcum at about 4% by weight. In certain embodiments, the formulation comprises colloidal silicon dioxide at about 3% by weight and hydrogenated castor oil at about 2%. In certain embodiments, the formulation comprises talcum at about 4% by weight and hydrogenated castor oil at about 2%. The use of hydrogenated castor oil has been found to be particularly advantageous in leading to formulations of amlodipine maleate having a pH as low as about 5.1 and having good stability.

The use of combinations of the above lubricants is also within the scope of the present invention. Accordingly, when a “lubricant” is referred to herein as being a component of a formulation, it is understood that the “lubricant” may actually be more than a single lubricant. For example, a combination of sodium stearyl fumarate and hydrogenated castor oil is contemplated as the lubricant of the present invention. Preferably such a combination of sodium stearyl fumarate and hydrogenated castor oil is present at a 1:1 ratio in the formulation, e.g., 1.5% by weight of sodium stearyl fumarate and 1.5% by weight of hydrogenated castor oil. By “combination,” as used above, is meant simply that both lubricants are present in the formulation. No particular interaction or physical relationship between the lubricants is implied. When more than one lubricant is present in the formulation, none of the lubricants present should contain magnesium.

The pH of the preferred formulation is preferably about 5.8 or lower. Preferred pH values are about 5.6, 5.5, 5.4, 5.3, 5.2, 5.1, and 5.0. Preferably, the pH is controlled without the use of acid addition. The pH of a formulation can be determined by measuring the pH of a 20% aqueous slurry of the formulation.

The results shown in the Examples herein demonstrate that those formulations that exclude lubricants containing magnesium, e.g., those formulations that exclude magnesium stearate or a mixture of magnesium stearate and another lubricant, produce less amlodipine aspartate. The Examples show that as the amount of magnesium in the formulation was decreased, the amount of amlodipine aspartate also decreased, with those formulations in which magnesium was completely eliminated showing the best results. While not intending to be bound by any particular interpretation, the inventors note that it may be the Mg²⁺ ion that is responsible for the effect of increased production of amlodipine aspartate since formulations comprising magnesium stearate show the effect while formulations comprising stearic acid do not. In view of this observation, the inventors expect that excluding other alkaline-earth metal ions, e.g., Ca²⁺, will also result in formulations of amlodipine maleate with improved stability, i.e., less amlodipine aspartate. Accordingly, the present invention includes formulations of amlodipine maleate where the formulations include a lubricant that does not contain alkaline-earth metal ions. In particular, the present invention includes formulations of amlodipine maleate where the formulations include a lubricant that does not contain calcium. In other embodiments, the formulations do not contain any excipients that introduce divalent alkaline-earth metal ions into the formulation.

The therapeutically effective amount of the pharmaceutical compositions of the present invention will generally comprise about 1 to 100 mg, preferably 1 to 25 mg, of amlodipine maleate administered from one to three times per day.

Amlodipine maleate can be made by methods known in the art. See, e.g., U.S. Pat. No. 4,572,909 and European Patent Application EP 089167. The form of amlodipine maleate used in the present invention may include anhydrates, solvates, hydrates, and partial hydrates as well as crystalline and amorphous forms. The ratio of amlodipine to maleate can be varied and can include the ratio of 1:1.

The present invention may be better understood by reference to the following non-limiting Examples, which are provided only as exemplary of the invention. The following examples are presented to more fully illustrate the preferred embodiments of the invention. They should in no way be construed, however, as limiting the broader scope of the invention.

### EXAMPLES

### Example 1

### Stability Studies with Magnesium Stearate

This example is a comparative stability study of a formulation containing magnesium stearate. Table 1 shows the results of a stability study with formulation 1150601, the contents of which are the same for formulation 1330203, described below.

TABLE 1
Batch No.: 1150601
Storage condition: 40° C./75% RH

| Time | Impurity D | Amlodipine aspartate |
|---|---|---|
| Initial | 0.3% | 0.08% |
| 1 month | 0.7% | 0.4% |
| 2 months | 1.0% | 0.6% |
| 3 months | 1.6% | 1.1% |

An important achievement of the present invention is to provide formulations having better stability at 40° C./75% RH than the above formulation.

Since 3 months (or even 1 month) is a long time for stability testing when developing a new formulation, a more rapid method was introduced: the batches were stored at 100° C. for 24 hours in an oven. The relative humidity was not controlled. The following results were obtained when a formulation with magnesium stearate was stressed under these conditions.

TABLE 2

| | | Amlodipine aspartate | |
|---|---|---|---|
| Batch No.: | Lubricant | Initial | Stressed |
| 1330203 | Magnesium stearate 1% | 0.09% | 6.5% |

The composition of Batch 1330203 is shown in Tables 5 and 8.

In view of the above results, it was considered desirable to develop formulations that contain a level of amlodipine aspartate at least below 5% after testing as above, i.e., 100° C. for 24 hours in an oven. Such formulations are useful in that they provide a more stable formulation than prior art formulations using magnesium stearate.

### EXAMPLE 2

### Effect of Individual Formulation Components on Production of Amlodipine Aspartate

During preliminary studies, it was found that the formation of amlodipine aspartate is increased with increasing temperature (this fact is supported by the poor stability data). Accordingly, an accelerated binary stability test was devised in which amlodipine maleate was mixed with individual formulation components and stored at 100° for 24 hours. Each formulation component was mixed with amlodipine maleate and tested in the absence of other formulation components. The ratio of amlodipine maleate to the formulation component was the same as in the preferred formulation shown in Table 3. Although not shown in Table 3, amlodipine maleate represents 3.21% by weight of the preferred formulation. Thus, e.g., microcrystalline cellulose was mixed with amlodipine maleate in the ratio of 63.79:3.21=19.87:1.

The results of the testing of the individual components are shown in Table 3. “Initial” refers to the percent of amlodipine aspartate before storage at 100° for 24 hours. “Stressed” refers to the percent of amlodipine aspartate after storage at 100° for 24 hours.

TABLE 3

| | | Amount of | |
|---|---|---|---|
| Formulation component tested | | amlodipine | |
| | Amount in the | aspartate | |
| | preferred | produced (% | |
| | formulation (% | by weight) | |
| Name | by weight) | Initial | Stressed |

| | | | |
|---|---|---|---|
| Microcrystalline cellulose | 63.79 | 0.05 | 1.16 |
| CaHPO₄, anhydrous | 30.00 | 0.05 | 0.27 |
| Sodium starch glycollate (type A) | 2.00 | 0.05 | 0.09 |
| Magnesium stearate | 1.00 | 0.05 | 5.50 |

As the results of the compatibility studies show, magnesium stearate was mainly responsible for the increase of the amount of amlodipine aspartate in the product.

### EXAMPLE 3

### Effect of Additional Lubricants on Production of Amlodipine Aspartate

Further binary studies with several other lubricants and combinations of lubricants were carried out. The execution of these experiments was as in Example 2. The results are shown in Table 4.

TABLE 4

| | | Amlodipine | |
|---|---|---|---|
| | | aspartate (%) | |
| Formulation component | | Initial | Stressed |
| I. | Magnesium stearate | <0.05 | 3.9 |
| II. | Dimeticone | <0.05 | <0.05 |
| III. | Magnesium stearate + Dimeticone (1:1) | <0.05 | 2.5 |
| IV. | Magnesium stearate + Dimeticone (4:1) | <0.05 | 3.9 |
| V. | Magnesium stearate + Macrogol 6000 (4:1) | <0.05 | 5.5 |
| VI. | Magnesium stearate + Hydrogenated castor oil | <0.05 | 3.7 |
| | (1:1) | | |
| VII. | Hydrogenated castor oil (0.5%) | <0.05 | 0.07 |
| VIII. | Hydrogenated castor oil (1.0%) | <0.05 | 0.08 |
| IX. | Sodium stearyl fumarate | <0.05 | 0.1 |
| X. | Stearic acid (1.0%) | 0.06 | 0.09 |
| XI. | Stearic acid (2.0%) | 0.06 | 0.1 |
| XII. | Dimeticone-Sodium stearyl fumarate (2.0%) | 0.06 | 0.1 |
| | (1:1) | | |
| XIII. | Dimeticone-Sodium stearyl fumarate (1.0%) | 0.06 | 0.09 |
| | (1:1) | | |

### EXAMPLE 4

### Effect of Different Lubricants on Production of Amlodipine Aspartate in Tablets Containing Complete Formulations

Tablets were manufactured in small scale with the above mentioned lubricants and lubricant combinations to test both stability and lubricant effect. The tablets contained all of the non-lubricant formulation components in Table 3 in the amounts shown in Table 3 plus different lubricants or combinations of lubricants as well as 3.21% by weight of amlodipine maleate. The powdered tablets were stored at 100° C. for 24 hours. Both initial and stressed samples were analyzed for impurities and degradation products. The results can be seen in Table 5

TABLE 5

| | | | Amlodipine | |
|---|---|---|---|---|
| | | Relevant binary | aspartate (%) | |
| Ch. No. | Lubricant used | study | Initial | Stressed |
| 1330203 | Mg. stearate 1.0% | I | 0.09 | 6.5 |
| 1260103 | Dimeticone 0.5% | III | <0.05 | 5.5 |
| | Mg. stearate 0.5% | | | |
| 1270103 | Mg. stearate 0.8% | IV | 0.05 | 7.3 |
| | Dimeticone 0.2% | | | |
| 1280103 | Mg. stearate 0.8% | V | 0.05 | 8.0 |
| | Macrogol 6000 | | | |
| 1290103 | Mg. stearate 0.5% | VI | 0.05 | 6.1 |
| | Hydrogenated | | | |
| | castor oil 0.5% | | | |
| 1300103 | Hydrogenated | VII | 0.05 | 3.0 |
| | castor oil 0.5% | | | |
| 1310103 | Hydrogenated | VIII | 0.05 | 2.1 |
| | castor oil 1.0% | | | |
| 1320103 | Sodium stearyl | IX | <0.05 | 1.5 |
| | fumarate | | | |
| 1350203 | Stearic acid 1% | X | 0.09 | 1.5 |
| 1360203 | Dimeticon 0.5% | XIII | 0.08 | 1.0 |
| | Sodium stearyl | | | |
| | fumarate 0.5% | | | |

When the results of the binary studies and the studies of the tablets are compared (see Table 6), it can be seen that the results with respect to lubricant are consistent from binary study to tablet study, and the amount of amlodipine aspartate after stress conditions is always higher in the complete formulation, i.e., tablet.

TABLE 6

| | Binary | Amlodipine | Ch. No. of | Amlodipine |
|---|---|---|---|---|
| Lubricant | study | aspartate (%) | tablet | aspartate (%) |
| Dimeticone 0.5% | III | 2.5 | 1260103 | 5.5 |
| Mg. stearate 0.5% | | | | |
| Dimeticone 0.2% | IV | 3.9 | 1270103 | 7.3 |
| Mg. stearate 0.8% | | | | |
| Mg. stearate 0.8% | V | 5.5 | 1280103 | 8.0 |
| Macrogol 6000 | | | | |
| Mg. stearate 0.5% | VI | 3.7 | 1290103 | 6.1 |
| Hydrogenated castor | | | | |
| oil 0.5% | | | | |
| Hydrogenated castor | VII | 0.07 | 1300103 | 3.0 |
| oil 0.5% | | | | |
| Hydrogenated castor | VIII | 0.08 | 1310103 | 2.1 |
| oil 1.0% | | | | |
| Sodium stearyl | IX | 0.1 | 1320103 | 1.5 |
| fumarate | | | | |
| Stearic acid 1% | X | 0.09 | 1350203 | 1.5 |
| Dimeticon 0.5% | XIII | 0.09 | 1360203 | 1.0 |
| Sodium stearyl | | | | |
| fumarate 0.5% | | | | |

### EXAMPLE 5

### Effect of Lubricants on Tablet Quality

Tablets were produced as in Example 4 and the behavior of the granule during tabletting was examined (see Table 7).

TABLE 7

| Ch. No. | Lubricant used | Observations during tabletting |
|---|---|---|
| 1260103 | Dimeticone 0.5% | good flowability, good compressibility |
| | Mg. stearate 0.5% | |
| 1270103 | Dimeticone 0.2% | good flowability, good compressibility |
| | Mg. stearate 0.8% | |
| 1280103 | Mg. stearate 0.8% | good flowability, good compressibility |
| | Macrogol 6000 | slightly sticking to the punches |
| 1290103 | Mg. stearate 0.5% | good flowability, good compressibility |
| | Hydrogenated castor | slightly sticking to the punches |
| | oil 0.5% | |
| 1300103 | Hydrogenated castor | good flowability, good compressibility |
| | oil 0.5% | sticking to the punches |
| 1310103 | Hydrogenated castor | good flowability, good compressibility |
| | oil 1.0% | sticking to the punches |
| 1320103 | Sodium stearyl | good flowability, good compressibility |
| | fumarate | |
| 1350203 | Stearic acid 1% | good flowability, good compressibility |
| | | sticking to the punches |
| 1360203 | Dimeticon 0.5% | good flowability, good compressibility |
| | Sodium stearyl | slightly sticking to the punches |
| | fumarate 0.5% | |

### EXAMPLE 6

### Effect of pH

An investigation of the effect of lowering the pH of the powdered tablets from Examples 4 and 5 from pH 5.8 was carried out. The pH was lowered without acid addition. Instead, a lower pH version of sodium starch glycollate (type B, rather than type A) was used. Small scale experimental batches of the tablets were made with sodium starch glycollate (type B) and with different lubricants. The effect of sodium starch glycollate (type B) on the pH of the powdered tablets can be seen in Table 8.

TABLE 8

| Ch. No. | Lubricant | pH |
|---|---|---|
| 1330203 | Magnesium stearate (1.0%) | 5.4 |
| 1340203 | Sodium stearyl fumarate (1.0%) | 5.4 |
| 1350203 | Stearic acid (1.0%) | 5.6 |
| 1360203 | Dimeticon 0.5% | 5.4 |
| | Sodium stearyl fumarate 0.5% | |

A comparison of sodium starch glycollate type A and sodium starch glycollate type B was made. The tablets were formulated with sodium stearyl fumarate as the only lubricant and either type A or type B sodium starch glycollate. The results for the amount of amlodipine aspartate can be seen in Table 9.

TABLE 9

| | | Amlodipine aspartate (%) | |
|---|---|---|---|
| Ch. No. | sodium starch glycollate | Initial | Stressed |
| 1320103 | Type A | <0.05 | 1.5 |
| 1340103 | Type B | 0.09 | 0.8 |

Lowering the pH by using type B sodium starch glycollate with the same lubricant decreased the amount of amlodipine aspartate.

### EXAMPLE 7

### Low pH Formulations

Certain formulations containing

| | |
|---|---|
| amlodipine maleate | 3% |
| microcrystalline cellulose | 57%-60% |
| low pH CaHPO₄, anhydrous | 32% |
| sodium starch glycollate (type B) | 2% |
| Silica colloidal, anhydrous or talcum | 0%-4% |
| a lubricant that does not contain magnesium | 1%-3% |

were prepared as tablets and were found to have low pH, and in some cases a very low pH of about 5.1. These low pH formulations were found to have good stability in that they had a low percentage of amlodipine aspartate after stress conditions (100° C./24 hours). The results are shown in Table 10.

TABLE 10

| | | amlodipine aspartate (%) | | |
|---|---|---|---|---|
| Ch. No. | Lubricant used | Initial | Stressed | pH |
| 1511203 | Sodium stearyl fumarate | 0.06% | 5.4% | 5.5 |
| | 2.5% | | | |
| 1521203 | Sodium stearyl fumarate | 0.07% | 4.0% | 5.6 |
| | 3% | | | |
| 1531203 | Silica colloidal, anhydrous | 0.06% | 3.3% | 5.6 |
| | 3% | | | |
| | Sodium stearyl fumarate | | | |
| | 3% | | | |
| 1541203 | Talcum 4% | 0.07% | 5.4% | 5.6 |
| | Sodium stearyl fumarate | | | |
| | 3% | | | |
| 1551203 | Silica colloidal, anhydrous | 0.06% | 2.2% | 5.5 |
| | 3% | | | |
| | Sodium stearyl fumarate | | | |
| | 2% | | | |
| | Dimeticone 1% | | | |
| 1561203 | Talcum 4% | 0.06% | 2.8% | 5.5 |
| | Sodium stearyl fumarate | | | |
| | 2% | | | |
| | Dimeticone 1% | | | |
| 1571203 | Silica colloidal, anhydrous | 0.06% | 2.3% | 5.1 |
| | 3% | | | |
| | Hydrogenated castor oil 2% | | | |
| 1581203 | Talcum 4% | 0.06% | 1.9% | 5.1 |
| | Hydrogenated castor oil 2% | | | |

Two batches (1551203 and 1571203) were put on stability at 40° C./75% RH. The one month results are found in Table 11.

TABLE 11

| | Impurity D | | Amlodipine aspartate | |
|---|---|---|---|---|
| | | 1 month at | | 1 month at |
| Batch No. | Initial | 40° C./75% RH | Initial | 40° C./75% RH |
| 1551203 | 0.3% | 3.7% | 0.06% | 0.2% |
| 1571203 | 0.3% | 6.5% | 0.06% | 0.4% |

It was unexpected that formulation 1571203 (which has a pH of 5.1, see Table 10) would contain such low levels of impurities (including amlodipine aspartate) as are shown in Table 11. This is because International Patent Publication WO 02/053134, at page 2, lines 22-28, teaches that the pH of amlodipine maleate formulations should be kept within the range of about 5.5 to 7.0, preferably 6.0 to 7.0, in order to minimize degradation reaction products such as amlodipine aspartate.

The invention having been described, it will be readily apparent to those skilled in the art that further changes and modifications in actual implementation of the concepts and embodiments described herein can easily be made or may be learned 5 by practice of the invention, without departing from the spirit and scope of the invention as defined by the following claims.

1. A formulation of amlodipine maleate where the formulation comprises a lubricant that does not contain alkaline-earth metal ions.

2. The formulation of claim 1, where the alkaline-earth metal ion is magnesium.

3. The formulation of claim 1, where the alkaline-earth metal ion is calcium.

4. A formulation of amlodipine maleate comprising:
a therapeutically effective amount of amlodipine maleate,
a binder,
a diluent,
a disintegrant, and
a lubricant that does not contain magnesium.

5. The formulation of claim 4, where the lubricant is selected from the group consisting of colloidal silicon dioxide, powdered cellulose, starch, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, macrogol 6000, dimeticone, stearic acid, and talcum.

6. The formulation of claim 5, where the lubricant is selected from the group consisting of sodium stearyl fumarate, dimeticone, macrogol 6000, hydrogenated castor oil, and stearic acid.

7. The formulation of claim 6, where the lubricant is hydrogenated castor oil.

8. The formulation of claim 6, where the lubricant is hydrogenated castor oil in combination with another lubricant.

9. The formulation of claim 8, where the other lubricant is talcum.

10. The formulation of claim 7, where the pH is about 5.1.

11. The formulation of claim 7, where the formulation comprises less than 0.5% amlodipine aspartate.

12. The formulation of claim 7, where the formulation comprises less than 0.5% amlodipine aspartate after storage at 40° C. and 75% relative humidity for one month.

13. The formulation of claim 4, where the formulation comprises less than 0.5% amlodipine aspartate.

14. The formulation of claim 4, where the formulation comprises less than 3% amlodipine aspartate after storage at 100° C. for 24 hours.

15. The formulation of claim 4, where the formulation comprises less than 0.5% amlodipine aspartate after storage at 40° C. and 75% relative humidity for one month.

16. The formulation of claim 4, where the pH is about 5.0 to 5.4.

17. The formulation of claim 16, where the pH is about 5.1.

18. The formulation of claim 4, where the binder is selected from the group consisting of acacia, alginic acid, carbomer, carboxymethylcellulose sodium, dextrin, ethyl cellulose, gelatin, guar gum, hydrogenated vegetable oil, hydroxyethyl cellulose, hydroxypropyl cellulose, hydroxypropyl methyl cellulose, liquid glucose, maltodextrin, methylcellulose, polymethacrylates, povidone, pregelatinized starch, sodium alginate, microcrystalline cellulose, modified cellulose, and starch.

19. The formulation of claim 18, where the binder is selected from the group consisting of microcrystalline cellulose, modified celluloses, and povidone.

20. The formulation of claim 4, where the diluent is selected from the group consisting of calcium hydrogen phosphate (CaHPO₄), anhydrous; lactose; and mannitol.

21. The formulation of claim 4, where the disintegrant is selected from the group consisting of alginic acid, carboxymethylcellulose calcium, carboxymethylcellulose sodium, croscarmellose sodium, crospovidone, guar gum, methyl cellulose, microcrystalline cellulose, polacrilin potassium, powdered cellulose, pregelatinized starch, sodium alginate, sodium starch glycolate type A, sodium starch glycolate B, and starch.

22. The formulation of claim 21, further where the disintegrant is selected from the group consisting of sodium starch glycollate (type A), sodium starch glycollate (type B), and crospovidone.

23. A formulation of amlodipine maleate comprising:
a therapeutically effective amount of amlodipine maleate
microcrystalline cellulose
calcium hydrogen phosphate (CaHPO₄), anhydrous
sodium starch glycollate (type B)
a lubricant that does not contain magnesium.

24. The formulation of claim 23, where the formulation comprises less than 0.5% amlodipine aspartate.

25. The formulation of claim 23, where the formulation comprises less than 3% amlodipine aspartate after storage at 100° C. for 24 hours.

26. The formulation of claim 23, where the formulation comprises less than 0.5% amlodipine aspartate after storage at 40° C. and 75% relative humidity for one month.

27. A formulation of amlodipine maleate comprising, by weight:

| | |
|---|---|
| amlodipine maleate | about 2%-4%% |
| microcrystalline cellulose | about 40%-70% |
| calcium hydrogen phosphate (CaHPO₄), anhydrous | about 25%-50% |
| sodium starch glycollate (type B) | about 1%-3% |
| a lubricant that does not contain magnesium | about 0.5%-7%. |

28. The formulation of claim 27, where the lubricant is two or more different lubricants that together represent about 0.5%-7% of the formulation, by weight.

29. The formulation of claim 27, where the lubricant is selected from the group consisting of colloidal silicon dioxide, powdered cellulose, starch, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, macrogol 6000, dimeticone, stearic acid, and talcum.

30. The formulation of claim 29, where the lubricant is selected from the group consisting of sodium stearyl fumarate, dimeticone, macrogol 6000, hydrogenated castor oil, and stearic acid.

31. The formulation of claim 30, where the lubricant is hydrogenated castor oil.

32. The formulation of claim 30, where the lubricant is hydrogenated castor oil in combination with another lubricant.

33. The formulation of claim 32, where the other lubricant is talcum.

34. The formulation of claim 31, where the pH is about 5.1.

35. A formulation of amlodipine maleate comprising, by weight:

| | |
|---|---|
| amlodipine maleate | 3.21% |
| microcrystalline cellulose | 59.79-63.79% |
| calcium hydrogen phosphate (CaHPO₄), anhydrous | 30.00% |
| sodium starch glycollate (type B) | 2-4% |
| a lubricant that does not contain magnesium. | 1-7% |

36. The formulation of claim 35, where the lubricant is selected from the group consisting of colloidal silicon dioxide, powdered cellulose, starch, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, macrogol 6000, dimeticone, stearic acid, and talcum.

37. The formulation of claim 36, where the lubricant is selected from the group consisting of sodium stearyl fumarate, dimeticone, macrogol 6000, hydrogenated castor oil, and stearic acid.

38. The formulation of claim 37, where the lubricant is hydrogenated castor oil.

39. The formulation of claim 35, where the lubricant is two or more different lubricants that together represent about 0.5%-7% of the formulation, by weight.

40. The formulation of claim 39, where the lubricant is hydrogenated castor oil in combination with another lubricant.

41. The formulation of claim 40, where the other lubricant is talcum.

42. The formulation of claim 38, where the pH is about 5.1.

43. A method of making a formulation of amlodipine maleate where the method comprises combining:
a therapeutically effective amount of amlodipine maleate
a diluent
a binder
a disintegrant
a lubricant that does not contain magnesium
where the resulting formulation of amlodipine maleate formed by so combining contains less than 0.5% amlodipine aspartate.

44. The method of claim 43, where the formulation comprises less than 3% amlodipine aspartate after storage at 100° C. for 24 hours.

45. The method of claim 43, where the formulation comprises less than 0.5% amlodipine aspartate after storage at 40° C. and 75% relative humidity for one month.

46. The formulation of claim 43, where the lubricant is two or more different lubricants that together represent about 0.5%-7% of the formulation, by weight.

47. The formulation of claim 43, where the lubricant is selected from the group consisting of colloidal silicon dioxide, powdered cellulose, starch, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, macrogol 6000, dimeticone, stearic acid, and talcum.

48. The formulation of claim 47, where the lubricant is selected from the group consisting of sodium stearyl fumarate, dimeticone, macrogol 6000, hydrogenated castor oil, and stearic acid.

49. The formulation of claim 48, where the lubricant is hydrogenated castor oil.

50. The formulation of claim 43, where the lubricant is hydrogenated castor oil in combination with another lubricant.

51. The formulation of claim 50, where the other lubricant is talcum.

52. The formulation of claim 49, where the pH is about 5.1.

53. A method of treating and/or preventing hypertension, angina, or heart failure comprising administering to a patient in need thereof a therapeutically effective amount of a pharmaceutical composition comprising:
amlodipine maleate,
a diluent,
a binder,
a disintegrant, and
a lubricant that does not contain magnesium,
where the pharmaceutical composition comprises less than 0.5% amlodipine aspartate.
